(19) United States
(12) Patent Application Publication
(10) Pub. No.: US 2025/0127740 A1
(43) Pub. Date: Apr. 24, 2025

(54) COMPOSITION CONTAINING 3-HYDROXYANTHRANILIC ACID AND COMPOUNDS THAT ALTER IRON HOMEOSTASIS AND METHOD OF ITS USE

(71) Applicant: ARIZONA BOARD OF REGENTS ON BEHALF OF THE UNIVERSITY OF ARIZONA, Tucson, AZ (US)

(72) Inventors: George SUTPHIN, Tucson, AZ (US); Luis ESPEJO, Tucson, AZ (US)

(21) Appl. No.: 18/708,126

(22) PCT Filed: Nov. 9, 2022
(86) PCT No.: PCT/US2022/049368

§ 371 Date: May 7, 2024

Related U.S. Application Data
(60) Provisional application No. 63/263,769, filed on Nov. 9, 2021.

(51) Int. Cl. A61K 31/196; A23B 2/762; A61K 31/164; A61K 33/26; A61K 47/54; A61P 31/04

(52) U.S. Cl. CPC A61K 31/196; A23B 2/762; A61K 31/164; A61K 33/26; A61K 47/542; A61P 31/04

(57) ABSTRACT

A composition containing 3-hydroxyanthanilic acid (3HAA) and a compound that alters iron homeostasis (an iron chelator or an iron supplement). The combination may be used as a new antibiotic drug. Combining 3HAA with iron chelation may have the dual benefit of inhibiting pathogen growth while repressing the pathological hyperinflammatory response in septic individuals. The combination may be effective at preserving food by preventing bacterial growth.

### STATEMENT REGARDING FEDERALLY FUNDED RESEARCH

This invention was made with government support under Grant No. GM133588 and GM136536 awarded by National Institutes of Health. The government has certain rights in the invention.

### RELATED APPLICATION

This application claims priority to U.S. Provisional Application 63/263,769 filed on Nov. 9, 2021, the content of which is incorporated herein by reference in its entireties for all purposes.

### BACKGROUND

Antibiotic resistance is one of the most prominent problems in healthcare today. According to the Centers for Disease Control and Prevention, the United States alone sees more than 2.8 million cases of antibiotic-resistant infection each year resulting in more than 35,000 deaths. Sepsis-dysfunctional systemic inflammatory response to infection leading to organ damage and failure-is a leading cause of mortality in intensive care units (ICUs) and among the most expensive health conditions treated in the United States. Sepsis survivors experience chronic inflammation, immune suppression, reduced physical function, elevated frailty, and higher mortality rates. The increasing prevalence of antibiotic resistance has left healthcare workers with a depleted toolbox to combat sepsis. Age is both a primary risk factor for developing sepsis and a major determinant of survival and long-term consequences. There is an urgent and growing need to identify novel antibiotics to combat both the general expansion of antibiotic-resistance bacterial pathogens and sepsis-particularly in elderly patients.

### SUMMARY

This disclosure provides a composition containing 3-hydroxyanthanilic acid (3HAA) and compounds that alter cellular iron homeostasis (e.g., an iron chelator or an iron supplement) as a new antibiotic drug. It is disclosed here that 3HAA sensitizes bacteria to changes in iron availability (both increasing iron media content through supplementation or chelation of iron). Iron is an important element that is necessary for many critical processes in biological systems, including both eukaryotic cells and bacteria. Iron chelators act by binding available iron, making it easier for cells and tissues to move and excrete. Combining 3HAA with an iron supplement or an iron chelator may have the dual benefit of killing and inhibiting growth of pathogenic bacteria while repressing the pathological hyperinflammatory response in septic individuals.

In one embodiment, provided here is a composition for inhibiting growth of certain bacterium, comprising 3HAA or a functional substitute thereof and an iron chelator. In one aspect, the iron chelator is selected from the group consisting of deferoxamine (DFO), deferasirox, deferiprone, desferrithiocin, quercetin, clioquinol, O-trensox, tachpyr, dexrazoxane, triapine, pyridoxal, di-2-pyridylketone thiosemicarbazones, flavan-3-ol, curcumin, apocynin, kolaviron, floranol, baicalein, baicalin, ligustrazine, epigallocatechin gallate, theaflavin, phytic acid, genistein, and combination thereof.

In another embodiment, a composition for inhibiting growth of certain bacterium comprises 3HAA or a functional substitute thereof and an iron supplement. In one aspect, the iron supplement is selected from the group consisting of ferric ammonium citrate (FAC), ferrous sulfate, ferrous bisglycinate, ferrous gluconate, ferrous citrate, ferric sulfate, iron dextran, iron sucrose, ferric gluconate, or combination thereof. In one aspect the composition will be given as a primary treatment.

In another embodiment, a composition for inhibiting growth of certain bacterium comprises 3HAA or a functional substitute thereof, the iron chelator and the iron supplement.

In one aspect, 3HAA is administered along with the iron chelator and/or the iron supplement. In another aspect, 3HAA alone, an iron supplement alone, or an iron chelator alone, is administered first to sensitize the bacterium, and the composition containing 3HAA and iron chelation or the composition containing 3HAA and iron supplementation is given subsequently, for example, after 1 day, 2 days, 3 days, 4 days, 5 days, 6 days or one week, or two weeks. In another aspect, 3HAA may be replaced with a functional substitute. By way of example only, the 3HAA functional substitute may include, another metabolite in the kynurenine metabolic pathway (e.g. 3-hydroxyanthranilc acid or cinnabarinic acid) or structural analogs or derivatives of 3HAA with similar molecular function.

In another embodiment, the molar ratio between 3HAA and the iron chelator or iron supplement is between 1000:1 and 1:1, between 100:1 and 20:1, between 80:1 and 40:1, or at about 65:1.

In another embodiment, a method is disclosed for inhibiting growth of certain bacterium by contacting the bacterium with a composition comprising 3-hydroxyanthanilic acid (3HAA) and an iron chelator or an iron supplement.

In another embodiment, the bacterium is selected from the group of genera consisting of Escherichia (including E. coli), Pseudomonas (including P. aeruginosa), Streptococcus (including S. pyogenes), Staphylococcus (S. aureus), Samonella, Legionella, Listeria, Bacillus, and Klebsiella (including K. pneumoniae and K. oxytoca).

In another embodiment, the disclosed composition is used for treating and/or preventing infection in a subject by administering to the subject an effective amount of the composition. In one aspect, the effective amount of the composition prevents reduces growth of pathogenic bacteria by 50%, 70%, 80%, 90%, 99%, or 100%. In another aspect, the effective amount of the composition prevents sepsis in the subject.

In another embodiment, the composition is administered to the subject by IV or oral administration. In one aspect, the dosage to be administered is between 1 mg/kg-body weight to 200 mg/kg/day 3HAA, between 0.1 mg and 75 mg/kg/day, or between 0.5 mg and 30 mg/kg/day iron chelator, and between 0.1 mg and 65 mg/kg/day, or between 0.5 mg and 30 mg/kg/day iron supplement.

In another embodiment, the disclosed composition is used for preventing food poisoning or enhancing food safety. In one aspect, an effective amount of the composition comprising 3-hydroxyanthanilic acid (3HAA) or a functional substitute and an iron chelator may be added to a food product to inhibit bacterial growth in the food product. In another aspect, the 3-hydroxyanthanilic acid (3HAA) or the functional substitute and an iron chelator or iron supplement are considered safe for consumer food product before being used on food products. Alternatively, in another aspect, any components not deemed safe for consumer food product are substantially or completely removed prior to sale or distribution of the food products to consumers.

In another embodiment, the 3HAA or the functional substitute thereof and iron supplements are natural products, but the iron chelator can be either a natural or synthetic product. In one aspect, the 3HAA or the functional substitute thereof is a metabolite produced by the kynurenine pathway from tryptophan. In one aspect, the iron chelator is synthetic. In another aspect, the 3HAA and the iron chelator are each present in the composition at a specific level in order for the 3HAA and the iron chelator to have a synergistic effect for inhibiting bacterial growth.

In one aspect, the food product is selected from the group consisting of meat, meat product, plant, prepackaged plant product, salad, fruits, and seafood.

### BRIEF DESCRIPTION OF THE FIGURES

The following figures form part of the present specification and are included to further illustrate aspects of the present invention.

FIG. 1 shows 3HAA Alone Significantly Negatively Impacts Bacterial Growth, but only at High Doses.

FIG. 2 shows that Deferoxamine Alone has Very Little Impact on Bacterial Growth.

FIG. 3 shows that FAC Alone does not Significantly Inhibit Bacterial Growth.

FIG. 4 shows that 3HAA/DFO Combination Synergistically Increases Growth Lag-Time.

FIG. 5 shows that 3HAA+DFO Combination Synergistically Reduces CFUs (E. coli).

FIG. 6 shows that 3HAA in combination with FAC does not impact CFUs (E. coli).

FIG. 7 shows that 3HAA+DFO or FAC and synergistically reduce colony size (E. coli).

FIG. 8 shows that 3HAA/DFO Combination Synergistically Reduces CFUs (P. aeruginosa).

FIG. 9 shows that 3HAA/DFO Combination Synergistically Reduces Colony Size (P. aeruginosa).

FIG. 10 shows that Pre-treatment with either 3HAA or DFO sensitizes bacteria to the combination.

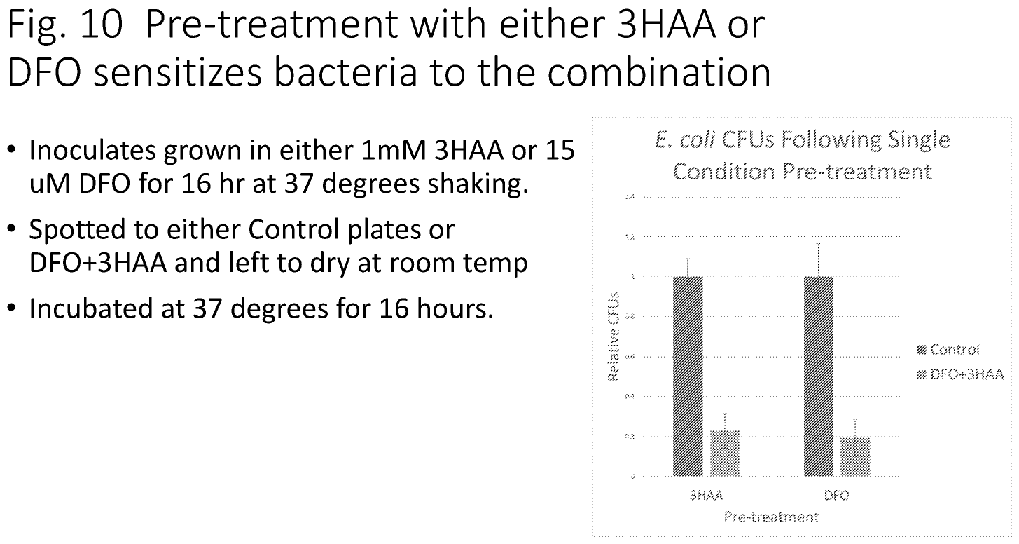

FIG. 11 shows that Combining 3HAA with altered iron homeostasis is not detrimental to host animals (C. elegans).

FIG. 12 shows that haao-1 worms treated with either FAC or DFO are protected from infection.

### DETAILED DESCRIPTION

The present disclosure provides a composition and methods for treating and/or preventing bacterial infection, treating and/or preventing sepsis or septic shock, or for enhancing food safety.

The term iron chelator refers to a compound or chemical that binds to iron and iron-containing compounds. Examples of iron chelators may include naturally existing or synthetic products. For example, many phytochemicals are iron chelators. See, e.g., Hatcher et al., Future Med Chem. 2009 December: 1 (9): 10.4155/imc.09.121.

The articles “a,” “an” and “the” are used to refer to one or more than one (i.e., to at least one) of the grammatical object of the article.

The terms “comprise”, “comprising”, “including” “containing”, “characterized by”, and grammatical equivalents thereof are used in the inclusive, open sense, meaning that additional elements are not expressly mentioned but may be included. It is not intended to be construed as “consists of only.”

The term “subject” or “patient” as used herein is intended to include animals.

Examples of subjects include but are not limited to mammals, e.g., humans, apes, monkeys, dogs, cows, horses, pigs, sheep, goats, cats, mice, rabbits, rats, and transgenic non-human animals. In an embodiment, the subject is a human.

The term “pathogen” or “infectious agent” is used herein to refer to any disease-causing virus, bacteria, fungi, protozoa, or parasite that infects and causes disease in a subject.

The term “contacting” is used synonymously with “incubating” and “exposing” and does not imply any specific time or temperature requirements unless otherwise indicated.

This disclosure can be further illustrated by the following Items:
Item 1. A composition for inhibiting growth of a bacterium, comprising
   a) 3-hydroxyanthanilic acid (3HAA) or a functional substitute thereof, and
   b) an iron chelator.
Item 2. The composition of Item 1, wherein the iron chelator is selected from the group consisting of deferoxamine (DFO), deferasirox, deferiprone, desferrithiocin, quercetin, clioquinol, O-trensox, tachpyr, dexrazoxane, triapine, pyridoxal, di-2-pyridylketone thiosemicarbazones, flavan-3-ol, curcumin, apocynin, kolaviron, floranol, baicalein, baicalin, ligustrazine, epigallocatechin gallate, theaflavin, phytic acid, genistein, and combination thereof.
Item 3. The composition of any of preceding items, further comprising an iron supplement selected from the group consisting of ferric ammonium citrate (FAC), ferrous sulfate, ferrous bisglycinate, ferrous gluconate, ferrous citrate, ferric sulfate, iron dextran, iron sucrose, ferric gluconate, and combination thereof.
Item 4. A composition for inhibiting growth of a bacterium, comprising
   (a) 3-hydroxyanthanilic acid (3HAA) or a functional substitute thereof, and
   (b) an iron supplement selected from the group consisting of ferric ammonium citrate (FAC), ferrous sulfate, ferrous bisglycinate, ferrous gluconate, ferrous citrate, ferric sulfate, iron dextran, iron sucrose, ferric gluconate, and combination thereof.
Item 5. The composition of any of preceding items, wherein the bacterium is selected from the group consisting of E. coli, P. aeruginosa Streptococcus pyogenes, and Klebsiella pneumoniae.
Item 6. The composition of any of preceding items, wherein the iron chelator is deferoxamine (DFO) and molar ratio between 3HAA and DFO is between 1000:1 and 1:1.
Item 7. A method for inhibiting growth of a bacterium, comprising contacting the bacterium with a composition comprising 3-hydroxyanthanilic acid (3HAA) and an iron chelator.
Item 8. The method of Item 7, wherein the bacterium is selected from the group consisting of E. coli, P. aeruginosa Streptococcus pyogenes, and Klebsiella pneumoniae .
Item 9. A method for treating or preventing infection in a subject, comprising administer to the subject an effective amount of the composition of Item 1.
Item 10. The method of Item 9, wherein the effective amount of the composition prevents sepsis.
Item 11. The method of any of Item 9 or 10, wherein the composition is administered by intravenous (IV) or oral administration.
Item 12. The method of any of Items 9-11, wherein the dosage to be administered is between 1 mg/kg-body weight to 200 mg/kg/day 3HAA, between 0.1 mg and 75 mg/kg/day, or between 0.5 mg and 30 mg/kg/day iron chelator, and between 0.1 mg and 65 mg/kg/day, or between 0.5 mg and 30 mg/kg/day iron supplement.
Item 13. The method of any of Items 9-12, wherein the iron chelator is deferoxamine (DFO) and molar ratio between 3HAA and DFO is between 1000:1 and 10:1.
Item 14. A method for preventing food poisoning or enhancing food safety, comprising contacting a food product with the composition of claim 1 to inhibit bacterial growth in the food product.
Item 15. The method of Item 14, wherein the food product is selected from the group consisting of meat, meat product, plant, prepackaged plant product, salad, fruits, and seafood.
Item 16. The composition of Item 1, wherein the 3HAA is a natural product, and the iron chelator is not a natural product.
Item 17. The composition of Item 1, wherein the 3HAA and the iron chelator are each present in the composition at a specific level in order for the 3HAA and the iron chelator to have a synergistic effect for inhibiting bacterial growth.

All references cited in this disclosure, including but not limited to patents, patent applications and published papers, are hereby incorporated by reference into this disclosure.

### EXAMPLES

The disclosure will now be illustrated with working examples, and which is intended to illustrate the working of disclosure and not intended to restrictively any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

### Example 1 3HAA Alone Significantly Negatively Impacts Bacterial Growth, but Only at High Doses

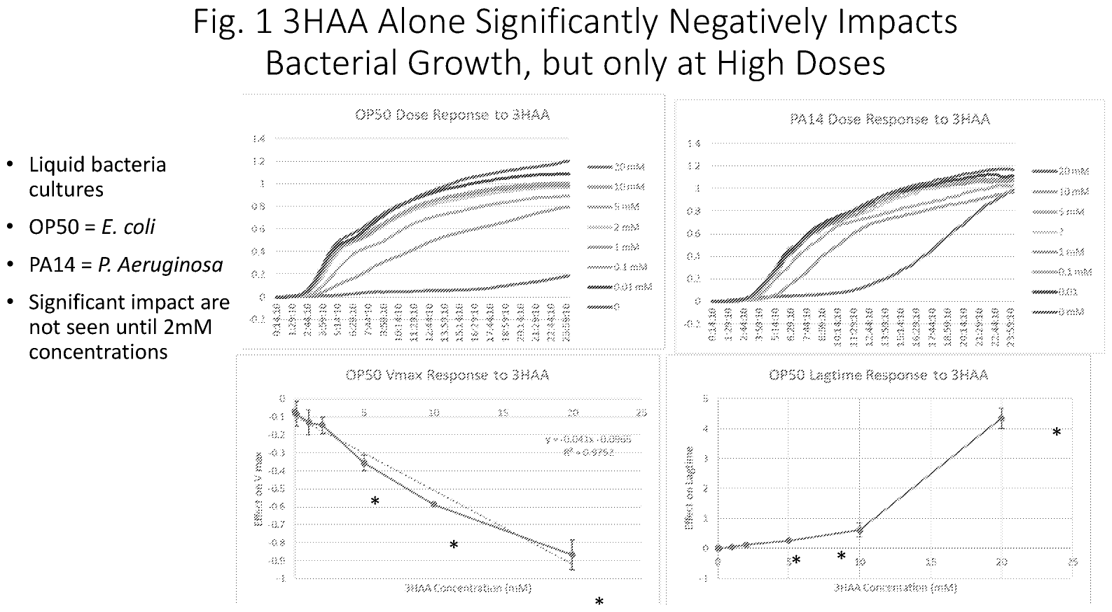

3HAA was tested for its ability to negatively impact bacterial growth. 3HAA was tested at different dosages for inhibition of E. coli and P. aeruginosa growth in liquid media (measured by optical density at 600 nm) and colony forming units (CFUs) on solid media. As shown in FIG. 1, 3HAA alone negatively affects growth of both E. coli and P. aeruginosa, but only at high doses.

### Example 2 Deferoxamine Alone has Very Little Impact on Bacterial Growth

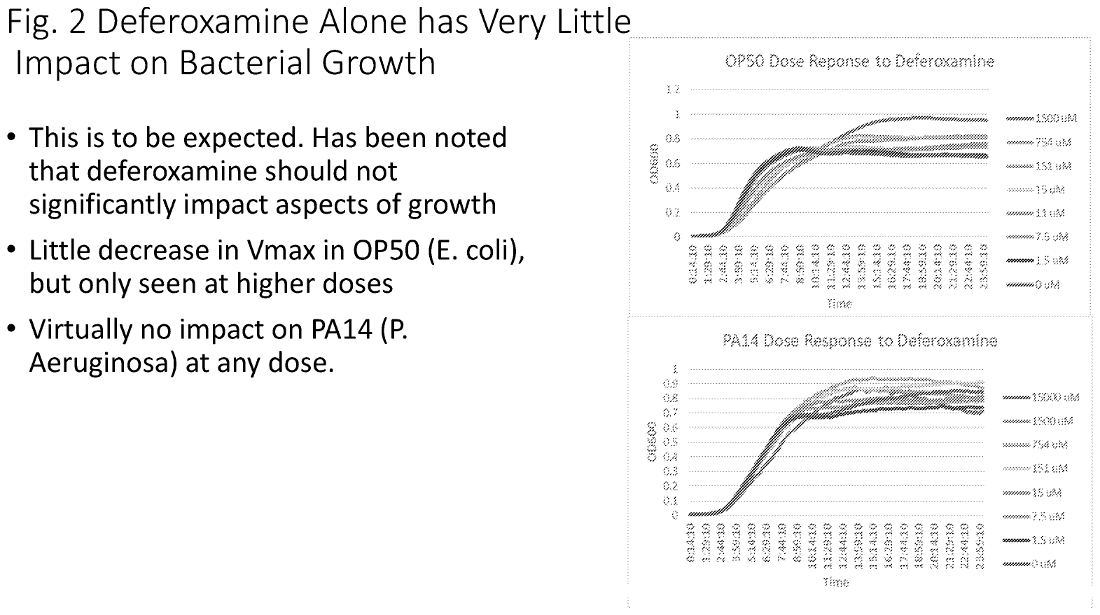

Deferoxamine (DFO) was also tested for its ability to impact bacterial growth. Deferoxamine was tested at different dosages for inhibition of E. coli and P. aeruginosa growth in liquid media (measured by optical density at 600 nm) and CFUs on solid media. As shown in FIG. 2, deferoxamine alone had very little impact on growth of either E. coli or P. aeruginosa.

### Example 3 FAC Alone Does not Significantly Inhibit Bacterial Growth

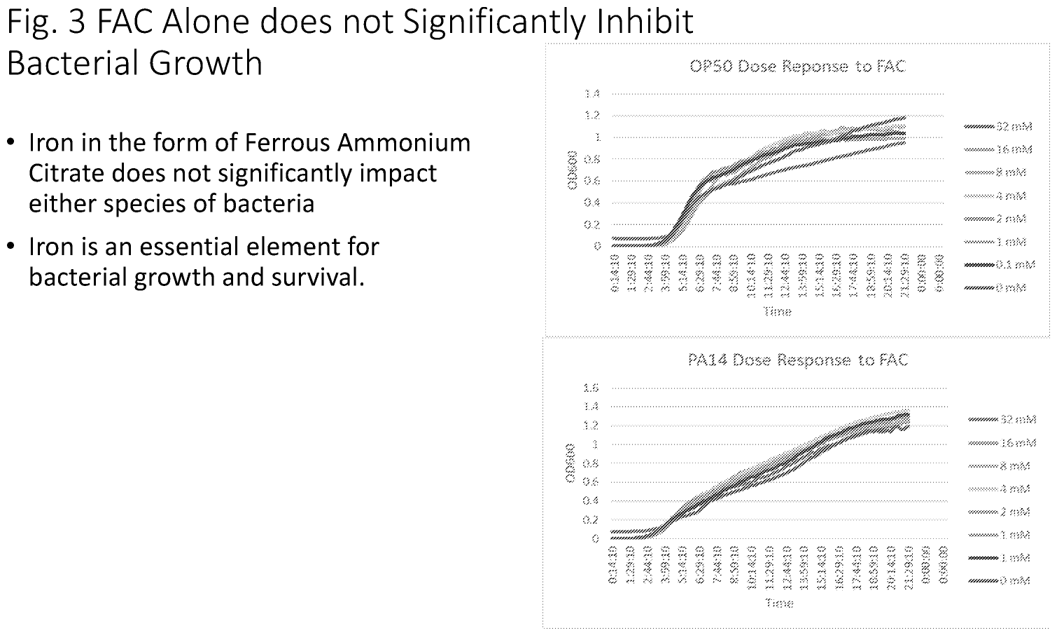

Iron in the form of ferric ammonium citrate (FAC) was tested for its ability to impact bacterial growth. FAC was tested at different dosages for inhibition of E. coli and P. aeruginosa growth in liquid media (measured by optical density at 600 nm) and CFUs on solid media. As shown in FIG. 3, FAC alone had very little impact on growth of either E. coli or P. aeruginosa.

### Example 4 3HAA and DFO Combination Synergistically Inhibits Bacterial Growth by Increasing Growth Lag-Time

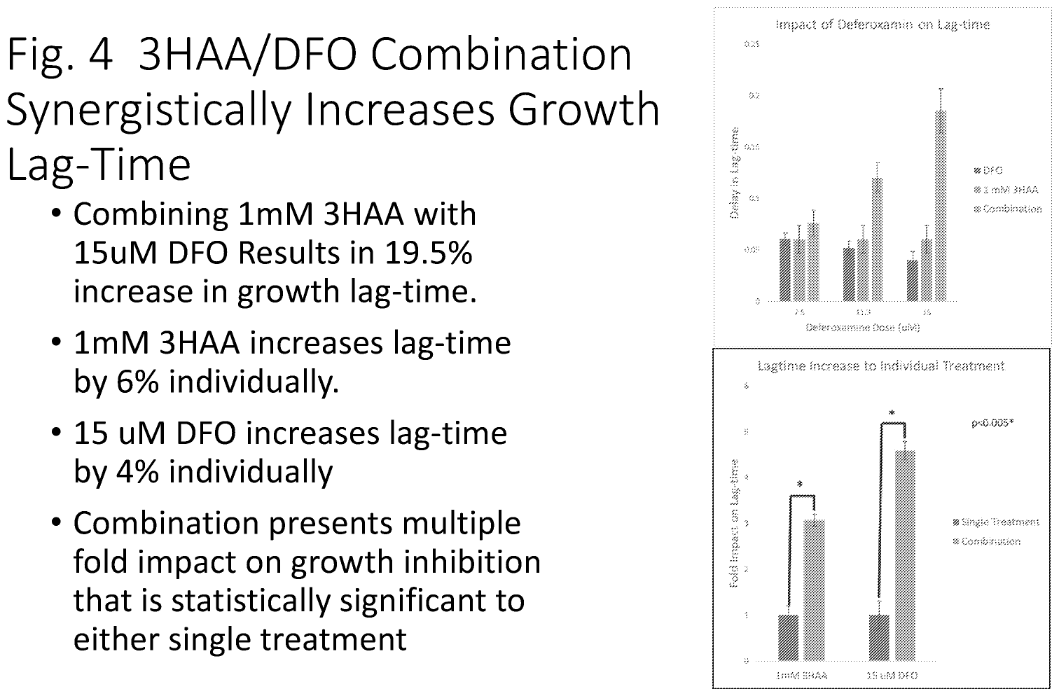

As shown in FIG. 4, combining 1 mM 3HAA with 15 uM DFO resulted in 19.5% increase in growth lag-time in liquid media. By contrast, 1 mM 3HAA alone increased lag-time by 6% individually. 15 uM DFO alone increased lag-time by 4% individually. Combination of 1 mM 3HAA with 15 uM DFO provided multiple fold impact on growth inhibition that is statistically significant to either single treatment alone.

### Example 5 3HAA and DFO Combination Synergistically Reduces CFUs (E. coli)

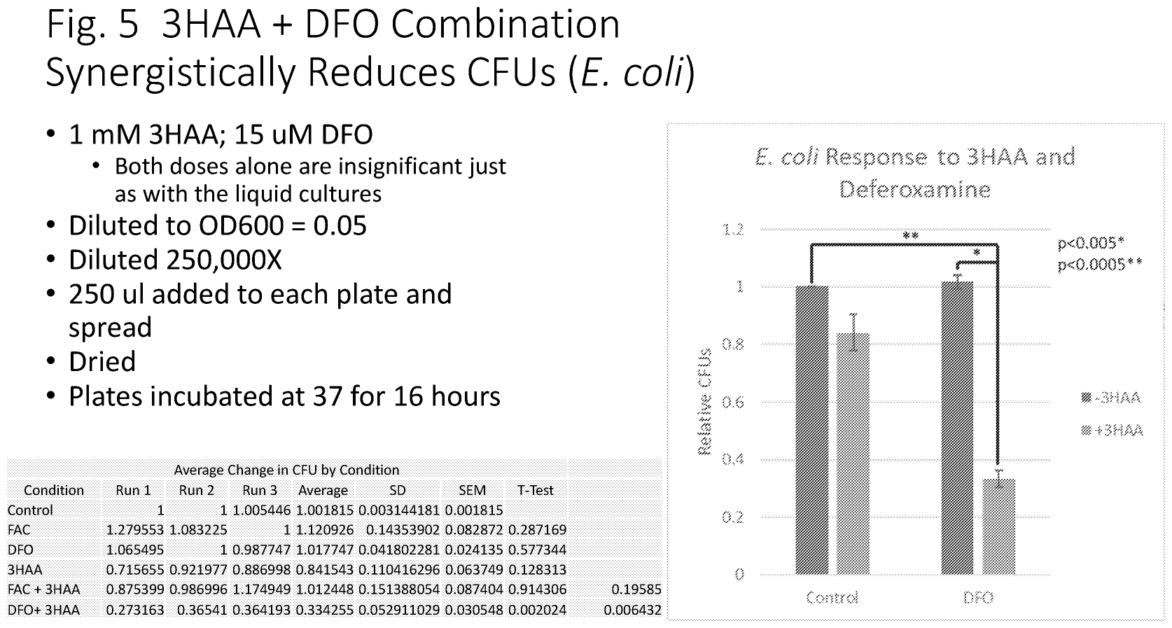

To test the effects of 3HAA and DFO alone and in combination on colony formation, 1 mM 3HAA; 15 uM DFO alone or in combination were added to culture plate containing E. coli. Neither 1 mM 3HAA nor 15 uM DFO alone showed significant impact. However, 1 mM 3HAA; 15 uM DFO together significantly inhibited colony formation by ˜65%, as shown in FIG. 5.

### Example 6 3HAA in Combination With FAC Does not Impact CFUs (E. coli)

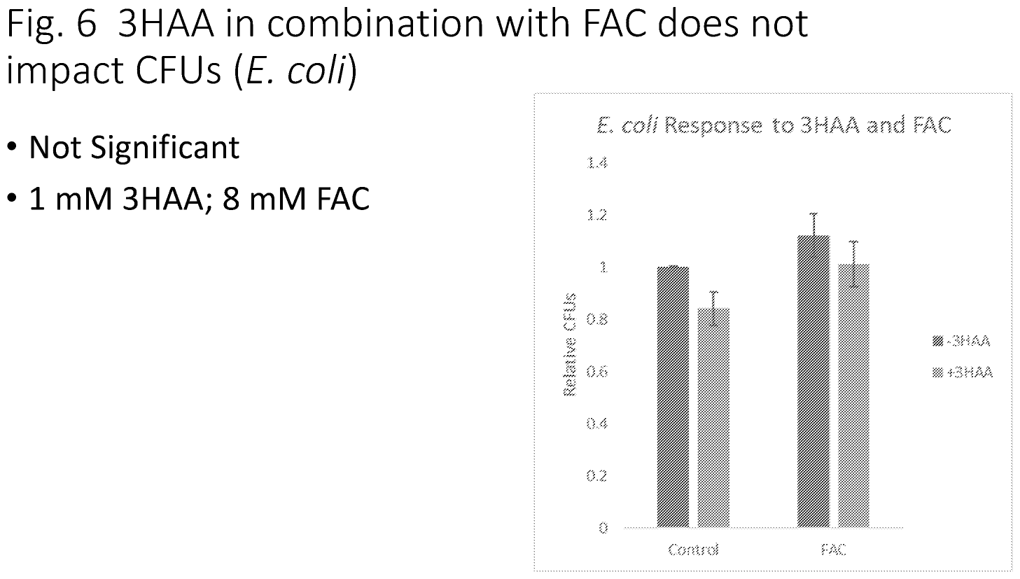

To test the effects of 3HAA and FAC on colony formation of E. coli, 1 mM 3HAA and 8 mM FAC alone or in combination were added to culture plate containing E. coli. As shown in FIG. 6, no significant impact was observed.

### Example 7 3HAA With Either DFO or FAC Synergistically Reduces Colony Size (E. coli)

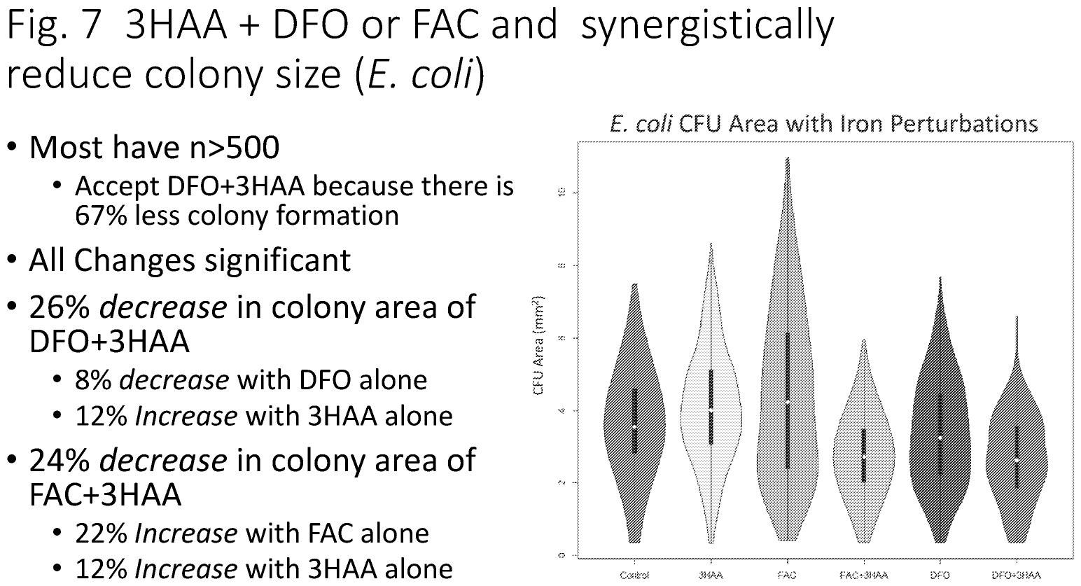

To test the effects of 3HAA, DFO and FAC on colony size, colony size was measured for E. coli in plates containing 1 mM 3HAA, 15 uM DFO, and 8 mM FAC alone and in combination. As shown in FIG. 7, 3HAA alone or FAC alone each increased colony size, while DFO alone decreased colony size by ˜8%. In contrast, DFO+3HAA decreased colony area by 26%, while FAC+3HAA decreased colony area by 24%, demonstrating synergistic toxicity of each combination to bacterial growth on solid media.

### Example 8 3HAA/DFO Combination Synergistically Reduces CFUs (P. aeruginosa)

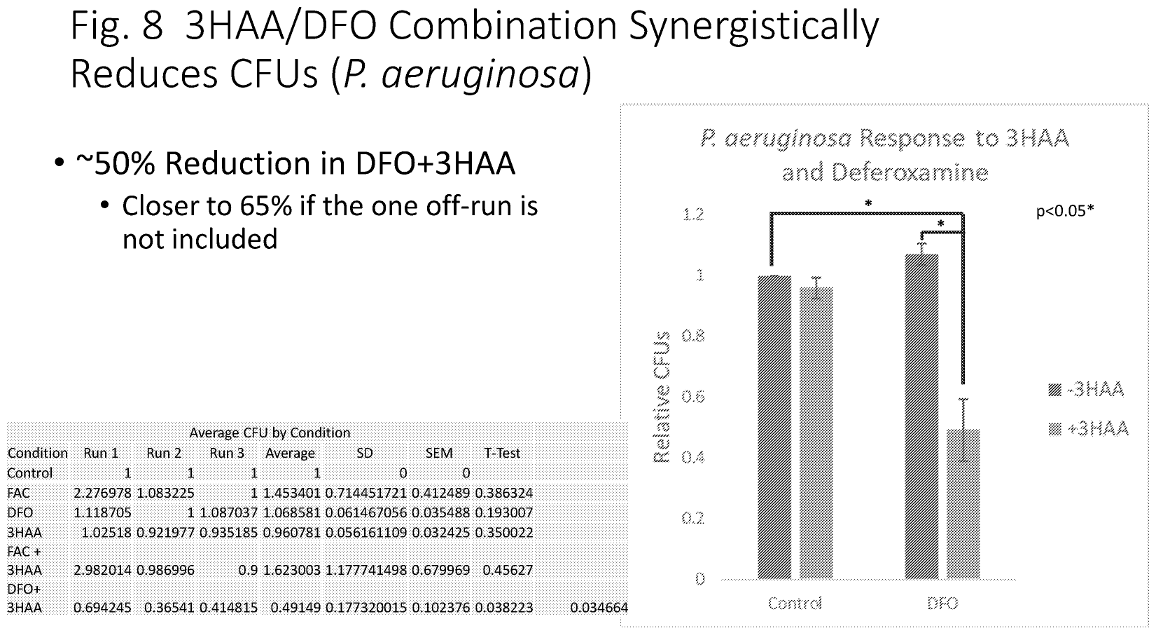

To test the effects of 3HAA and DFO on colony formation of P. aeruginosa, 1 mM 3HAA and 15 uM DFO alone or in combination were added to culture plate containing P. aeruginosa. As shown in FIG. 8, 1 mM 3HAA and 15 uM DFO together significantly inhibited colony formation of P. aeruginosa, while neither affected colony formation alone, demonstrating synergy.

### Example 9 3HAA with either DFO or FAC synergistically reduce colony size (P. aeruginosa)

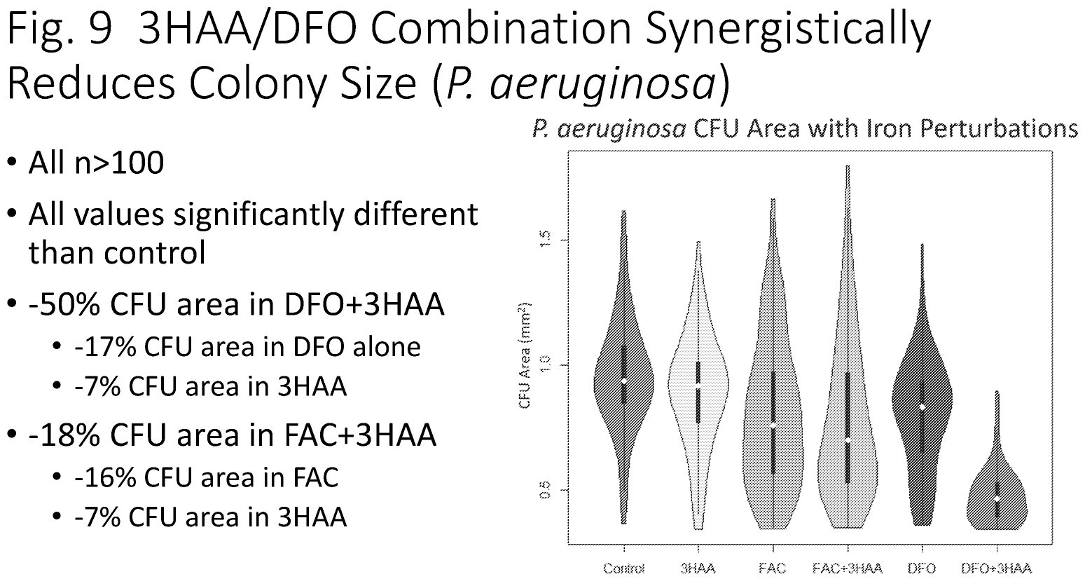

To test the effects of 3HAA, DFO and FAC on colony size, colony size was measured for P. aeruginosa in plates containing 1 mM 3HAA, 15 uM DFO, and 8 mM FAC alone and in combination. As shown in FIG. 9, DFO+3HAA decreased colony area by 50%, while FAC+3HAA decreased colony area by 18%, in both cases significantly more than the corresponding single-compound treatments.

### Example 10 Pre-Treatment With Either 3HAA or DFO Sensitizes Bacteria to the Combination

E. coli inoculates were grown in liquid media containing either control (no additives), 1 mM 3HAA or 15 uM DFO for 16 hr at 37 degrees with shaking. The cultures were spotted to either Control plates (no additives) or plate containing DFO+3HAA and left to dry at room temperature. After incubated at 37 degrees for 16 hours, colonies were counted. The effect of 3HAA and DFO in combination was more pronounced for the single pretreatment plate as compared to the no pretreatment control, suggesting that pretreatment with either 3HAA or DFO sensitizes bacteria to the combination of 3HAA and DFO.

### Example 11 Combining 3HAA With Altered Iron Homeostasis is not Detrimental to Host Animals (C. elegans)

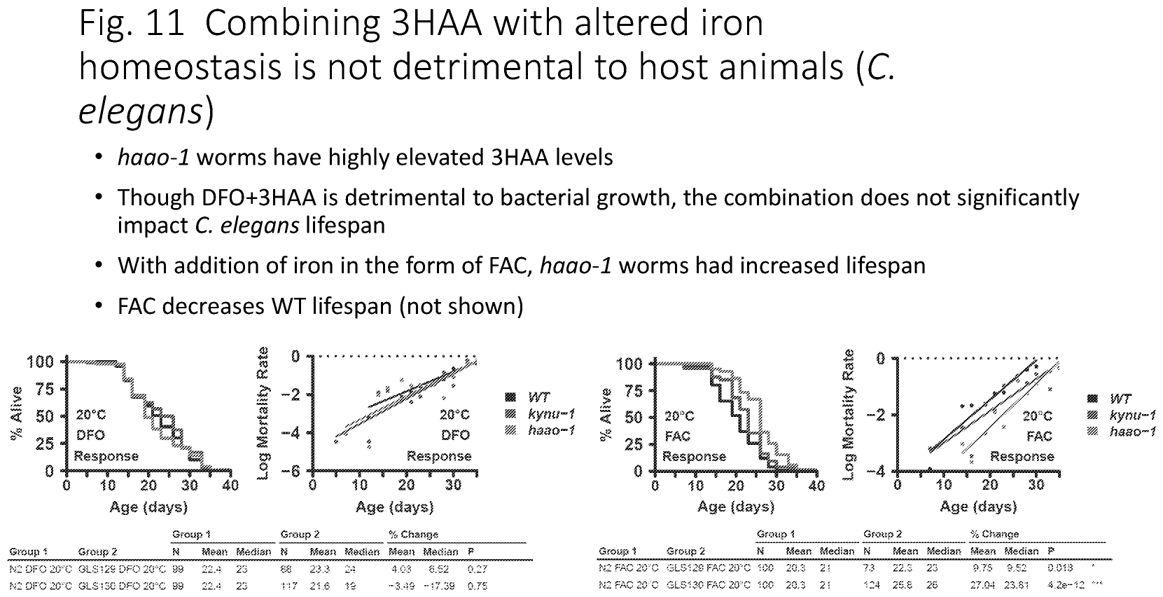

To determine if 3HAA in combination with DFO or FAC have any toxic effects to an animal, DFO was added to culture medium of haao-1 knockout worms, which have elevated endogenous 3HAA. We chose this approach because adding 3HAA and DFO or FAC to the growth media would impact bacterial growth, which can itself impact worm survival. Using the haao-1 knockout strain exposes the animals, but not their bacterial food source, to 3HAA. As shown in FIG. 11, neither the combination of DFO and elevated 3HAA (haao-1) nor the combination of FAC with elevated 3HAA (haao-1) were toxic to C. elegans relative to wild type animals.

### Example 12 haao-1 Worms Treated With Either FAC are Protected From E. coli Infection

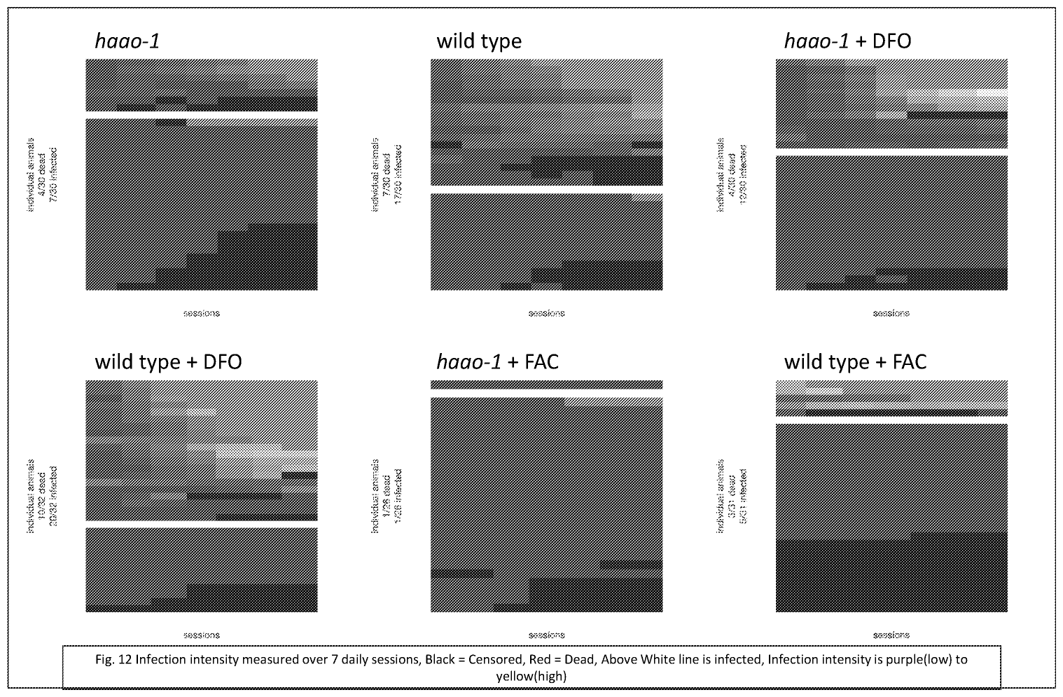

To determine if worms treated with either FAC or DFO are protected from infection, 30 individual C. elegans were challenged with fluorescently labeled E. coli bacteria. Infection was monitored using fluorescence microscopy over 7 days. Heatmap of infection progression of individual animals over days—purple 32 not infected, red=high infection. As shown in FIG. 12, haao-1 knockout worms (high endogenous 3HAA) and FAC treated wild type worms were protected from infection. This protection was enhanced in haao-1 knockout worms treated with FAC.

### REFERENCES

The following references, along with those cited throughout this disclosure, are hereby incorporated by reference into this disclosure.

Hatcher et al., Synthetic and natural iron chelators: therapeutic potential and clinical use. Future Med Chem. 2009 December: 1 (9): 10.4155/fmc.09.121.

1. A composition for inhibiting growth of a bacterium, comprising
a) 3-hydroxyanthanilic acid (3HAA) or a functional substitute thereof, and
b) an iron chelator.

2. The composition of claim 1, wherein the iron chelator is selected from the group consisting of deferoxamine (DFO), deferasirox, deferiprone, desferrithiocin, quercetin, clioquinol, O-trensox, tachpyr, dexrazoxane, triapine, pyridoxal, di-2-pyridylketone thiosemicarbazones, flavan-3-ol, curcumin, apocynin, kolaviron, floranol, baicalein, baicalin, ligustrazine, epigallocatechin gallate, theaflavin, phytic acid, genistein, and combination thereof.

3. The composition of claim 1, further comprising an iron supplement selected from the group consisting of ferric ammonium citrate (FAC), ferrous sulfate, ferrous bisglycinate, ferrous gluconate, ferrous citrate, ferric sulfate, iron dextran, iron sucrose, ferric gluconate, and combination thereof.

4. A composition for inhibiting growth of a bacterium, comprising
(a) 3-hydroxyanthanilic acid (3HAA) or a functional substitute thereof, and
(b) an iron supplement selected from the group consisting of ferric ammonium citrate (FAC), ferrous sulfate, ferrous bisglycinate, ferrous gluconate, ferrous citrate, ferric sulfate, iron dextran, iron sucrose, ferric gluconate, and combination thereof.

5. The composition of claim 1, wherein the bacterium is selected from the group consisting of E. coli, P. aeruginosa Streptococcus pyogenes, and Klebsiella pneumoniae.

6. The composition of claim 2, wherein the iron chelator is deferoxamine (DFO) and molar ratio between 3HAA and DFO is between 1000:1 and 1:1.

7. A method for inhibiting growth of a bacterium, comprising contacting the bacterium with a composition comprising 3-hydroxyanthanilic acid (3HAA) and an iron chelator.

8. The method of claim 7, wherein the bacterium is selected from the group consisting of E. coli, P. aeruginosa Streptococcus pyogenes, and Klebsiella pneumoniae.

9. A method for treating or preventing infection in a subject, comprising administer to the subject an effective amount of the composition of claim 1.

10. The method of claim 9, wherein the effective amount of the composition prevents sepsis.

11. The method of claim 9, wherein the composition is administered by intravenous (IV) or oral administration.

12. The method of claim 9, wherein the dosage to be administered is between 1 mg/kg-body weight to 200 mg/kg/day 3HAA, between 0.1 mg and 75 mg/kg/day, or between 0.5 mg and 30 mg/kg/day iron chelator, and between 0.1 mg and 65 mg/kg/day, or between 0.5 mg and 30 mg/kg/day iron supplement.

13. The method of claim 9, wherein the iron chelator is deferoxamine (DFO) and molar ratio between 3HAA and DFO is between 1000:1 and 10:1.

14. A method for preventing food poisoning or enhancing food safety, comprising contacting a food product with the composition of claim 1 to inhibit bacterial growth in the food product.

15. The method of claim 14, wherein the food product is selected from the group consisting of meat, meat product, plant, prepackaged plant product, salad, fruits, and seafood.

16. The composition of claim 1, wherein the 3HAA is a natural product, and the iron chelator is not a natural product.

17. The composition of claim 1, wherein the 3HAA and the iron chelator are each present in the composition at a specific level in order for the 3HAA and the iron chelator to have a synergistic effect for inhibiting bacterial growth.
